(19) United States
(12) Patent Application Publication
(10) Pub. No.: US 2012/0094989 A1
(43) Pub. Date: Apr. 19, 2012

(54) 5-HT RECEPTOR MODULATING COMPOUNDS

(75) Inventors: Jo Klaveness, Oslo (NO); Bjame Brudeli, Oslo (NO); Finn Olav Levy, Oslo (NO); Lise Roman Moltzau, Nittedal (NO); Trygve Gulbrandsen, Kolsaas (NO)

(73) Assignee: SERODUS AS, Oslo (NO)

(21) Appl. No.: 13/262,356

(22) PCT Filed: Apr. 1, 2010
(86) PCT No.: PCT/GB2010/000656

§ 371 Date: Dec. 15, 2011

(30) Foreign Application Priority Data
Apr. 1, 2009 (GB) 0905641.7

(51) Int. Cl. A61K 31/5365; C07D 211/58; C07D 401/10; C07D 211/34; A61P 13/00; A61K 31/454; A61K 31/445; A61P 9/00; A61P 1/00; C07D 498/04; A61K 31/4468

(52) U.S. Cl. 514/230.2; 544/89; 546/224; 546/210; 546/234; 514/329; 514/326; 514/331

(57) ABSTRACT

The present invention relates to compounds having 5-hydroxytryptamine receptor modulating activity, in particular compounds having an acidic moiety held distant from the 5-HT pharmacophore by a rigid linker group, to compositions containing such compounds and methods of treatment using them. Such compounds have an increased affinity for the 5-HT receptor and a reduced hERG effect. Certain compounds of the invention further exhibit an angiotensin II receptor modulating activity. Claimed are compounds of formula (I): HT-L-A. HT is a 5-HT receptor modulating moiety containing a basic nitrogen atom; A is an acid moiety; L is a linker moiety.

This invention relates to compounds having 5-hydroxytryptamine (hereinafter “5-HT”) receptor modulating activity, in particular compounds having an acidic moiety held distant from the 5-HT pharmacophore by a linker group so as to prevent the acidic moiety and the 5-HT pharmacophore on the same molecule from interacting. The invention also relates to prodrugs and salts of the modulator compounds and to compositions comprising these compounds, salts and prodrugs.

Serotonin (5-hydroxytryptamine or 5-HT) is a monoamine neurotransmitter. Serotonin is active in the central nervous system (CNS), demonstrating a broad activity in the brain in particular, and also in the gastrointestinal tract where it stimulates vomiting.

A number of receptor families and sub-families have been identified which are modulated by serotonin, these being known as 5-HT or 5-HTₓ receptors. Certain 5-HTₓ receptor subtypes are found within the CNS, e.g. 5-HT_{1A}, 5-HT_{5A} and 5-HT₆, whereas others are found outside the CNS, e.g. 5-HT_{2B}. Some receptor subtypes are found on both sides of the blood-brain barrier, e.g. 5-HT₄, where they potentiate different effects in the different locations.

Modulators (i.e. agonists or antagonists) of 5-HT receptors have been shown to be useful in the treatment of a wide range of conditions and are used as antidepressants, anxiolytics, antiemetics, antipsychotics and anti-migraine agents.

Many naturally-occurring and synthetic compounds are known which have a modulatory activity towards the 5-HT receptors. In particular, both agonists and antagonists of most receptors are known. For example, agonists of the 5-HT₄ receptor include cisapride, metoclopramide, renzapride and tegaserod, whereas one antagonist of the 5-HT₄ receptor is piboserod. Piboserod is a selective 5-HT₄ receptor antagonist used for the management of atrial fibrillation and irritable bowel syndrome, and has the following molecular structure:

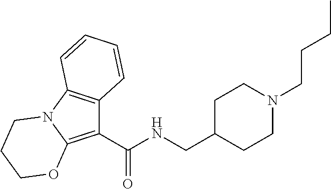

In this structure, the 5-HT pharmacophore includes a basic nitrogen atom (in the piperidine ring) which is substituted by an n-butyl chain.

WO 2007/007072 describes how the specificity of action of 5-HT receptor modulators may be enhanced by attaching an acid moiety to the 5-HT pharmacophore via a linking group. This modification hinders passage of the modulator across the blood-brain barrier and thus restricts the effects of an administered modulator to the side of the barrier on which it is administered. Examples of acid: 5-HT pharmacophore constructs are given in WO 2007/007072, as well as in WO 2007/149929 and WO 2005/061483.

The majority of the acid:5-HT pharmacophore constructs disclosed in these publications involve a readily flexible linker between the acid group and the basic nitrogen atom within the pharmacophore, for example a pentamethylene group as in Example 50 in WO 2007/007072 or a dimethyleneaminomethyl-p-phenylene group as in Compound 23 in WO 2007/149929.

The present inventors, however, have found that the overall performance of such acid: 5-HT pharmacophore constructs is improved if the linker between the acid group (or its precursor if in prodrug form) and the basic nitrogen atom in the pharmacophore serves to maintain a distance between the two of several Å (0.1 nm; 10⁻¹⁰ m). In particular, the resultant compounds display increased binding affinities for their receptors. This distancing of the basic nitrogen and the acid group may readily be achieved by the use of linkers which, in part at least, are rigid, or which are substituted by bulky substituents preventing rotation. Rigidity can be achieved by, for example, incorporation of cyclic groups, especially unsaturated groups, of fused rings, bridged rings or bonds which on rotation do not bring the nitrogen and acid close together. WO 2007/007072, WO 2005/061483 and WO 2007/149929 make no suggestion that low flexibility in the groups linking the basic nitrogen and the acid group is important or desirable; however WO 2005/061483 and WO 2007/007072 do describe some compounds which utilise a methylene-p-phenylene linker.

The compounds of the present invention are particularly and surprisingly advantageous over the compounds of the prior art. Particular advantages include one or more of the following: increased affinity for the 5-HT receptor, believed to be because the acidic proton cannot interfere with the active basic nitrogen atom of the pharmacophore; and a reduced blocking effect on the human ether-a-go-go related gene (hERG) channels. This reduced hERG effect is a critical parameter for consideration of the toxicological effects of the compounds of the invention; hERG blocking activity is linked to ventricular arrythmias and sudden death in the clinical setting. Further advantages of the preferred compounds of the invention include one or more of the following: a greater selectivity of modulation of the peripheral 5-HT receptors, especially the 5-HT₄ receptors; an antagonistic effect on angiotensin II receptors; little or no central nervous system toxicity effects when using clinically effective doses; high affinity for 5-HT receptors and thus a lowered clinical dose; and ease of preparation.

Thus viewed from one aspect the invention provides a 5-HT receptor modulator being a compound of formula I:

HT-L-A (I)

(wherein HT is a 5-HT receptor modulating moiety (“the 5-HT pharmacophore”) containing a basic nitrogen atom;
A is an acid moiety; and
L is a linker moiety serving to maintain said basic nitrogen atom and said acid moiety at a separation of at least 0.4 nm, preferably at least 0.5 nm, more preferably at least 0.6 nm, especially at least 0.65 nm, e.g. up to 2 nm) or a prodrug form or salt thereof.

In one embodiment, the compounds of formula I are other than HT-CH₂-p-phenylene-A.

Preferred prodrugs of the acidic moiety include esters and amides of carboxylic acids, especially methyl esters thereof, and N-aryl derivatives of tetrazoles, especially N-triphenylmethyl derivatives thereof. Typical esters include alkyl esters, substituted alkyl esters, aryl esters, substituted aryl esters and acyloxyalkyl esters. Substituent groups which may be present include straight-chained, branched and cyclic alkyl groups. Such groups may be saturated or unsaturated and may further be interrupted by one or more heteroatoms selected from oxygen, sulphur and nitrogen. The substituent groups may further contain one or more carbonyl or thiocarbonyl groups. Preferred substituents include C₁₋₆-alkyl (e.g. methyl) groups. Other preferred substituents include heterocyclic rings containing one or more oxygen atoms, and optionally at least one carbonyl group. Examples of such groups include 1-3-dioxolane and 1,3-dioxol-2-one.

Preferred salts of the compounds of the invention are pharmaceutically acceptable salts, including sodium, potassium, magnesium and ammonium salts thereof as well as salts with anions such as chloride, sulphate and carbonate.

In a preferred embodiment, HT denotes a moiety having an affinity for the 5-HT₄ receptor subgroup, e.g. a 5-HT₄ receptor-specific moiety, especially preferably a moiety with 5-HT₄ antagonist activity.

Examples of suitable HT groups include those of formula II:

Ar—(C(O))ₙ-(E)ₘ-(G)ₚ-BN— (II)

(wherein Ar is an optionally substituted aryl ring optionally fused with one or more rings selected from: non-aromatic, optionally substituted, carbocylic rings; non-aromatic heterocyclic rings; carbocyclic aromatic rings; and heteroaromatic rings;
n is 0 or 1, preferably 1;
m is 0 or 1, preferably 1;
E is O or NH;
p is 0 or 1, preferably 1;
G is a C₁₋₆-alkyl, C₃₋₇-cycloalkyl, C₁₋₆-alkyl-C₃₋₇-cycloalkyl or C₃₋₇-cycloalkyl-C₁₋₆-alkyl group; and
BN is a basic nitrogen moiety, preferably a moiety selected from an amine group, an amide group, a carbamate or a carbamate derivative, urea or a urea derivative, a carbazimidamide, a nitrogen-containing heterocyclic ring, a nitrogen-containing heteroarylic ring, and an azabicyclic ring).

As used herein, the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Illustrative examples of “aryl” rings include optionally substituted phenyl, naphthalenyl, phenanthrenyl, anthracenyl, tetralinyl, fluorenyl, indenyl and indanyl. A preferred aryl group is phenyl. The term “aryl” relates to aromatic, preferably benzenoid groups connected via one of the ring-forming carbon atoms, and optionally carrying one or more substituents selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. As stated, preferred aryl groups are phenyl and, most suitably, substituted phenyl groups carrying one or two of the substituents listed above which may be the same or different.

Other preferred examples of suitable aryl groups include optionally substituted benzyl, naphthalene, indoline, indole, oxazinoindoline, indolizine, isoindoline, indene, indane, indazole, azulene, benzimidazole, benzofuran, benzothiophene, benzthiazole, purine, 4H-quinolizine, quinoline, isoquinoline, cinnoline, phthalazine, quinazoline, quinoxaline, 1,3-naphthyridine, pteridine, coumaran, benzodioxane, benzopyran, chroman, isochroman, carbazole, acridine, phenazine, phenothiazine, phenoxazine, thianthrene, phenanthrene, anthracene, tetralin, fluorene, and acenaphthylene, each of which may be optionally substituted. More preferably, the aryl group may be selected from benzyl, naphthalene, indole, benzodioxane, indazole and oxazinoindole.

The term “heterocyclic ring” is intended to mean three-, four-, five-, six-, seven- and eight-membered rings wherein carbon atoms together with from 1 to 3 heteroatoms constitute said ring. A heterocyclyl may optionally contain one or more unsaturated bonds situated in such a way, however, that an aromatic pi-electron system does not arise. The heteroatoms are independently selected from oxygen, sulphur and nitrogen. A heterocyclic ring may further contain one or more carbonyl or thiocarbonyl functionalities, so as to make the definition include oxo-systems and thio-systems such as lactams, lactones, cyclic imides, cyclic thioimides, cyclic carbamates, and the like. Heterocyclic rings may optionally-also be fused to aryl rings, such that the definition includes bicyclic structures. Preferred such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Illustrative examples of “heterocyclic rings” are the heterocycles tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, thiazoline, thiazolidine and 1,3-oxathiolane. Binding to the heterocycle may be at the position of a heteroatom or via a carbon atom of the heterocycle, or, for benzo-fused derivatives, via a carbon of the benzenoid ring.

The basic nitrogen moiety (BN) may be any array of organic forms of nitrogen. Suitable forms of the basic nitrogen moiety may be selected from the group comprising an amine group, amide group, carbamates and urea derivatives, carbazimidamides, a nitrogen-containing heterocyclic or heteroarylic ring, including azabicycles. Amine groups can be primary, secondary or tertiary amines. Suitable nitrogen-containing heterocyclic or heteroaryl include pyridyl (pyridinyl), pyrimidinyl, thiazolyl, pyrazolyl, imidazolyl, tetrazolyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydroquinolinyl, tetrahydroisoquinolinyl, decahydroquinolinyl or octahydroisoquinolinyl, azocinyl, triazinyl, 6H-1,2,5-thiadiazinyl, 2H,6H-1,5,2-dithiazinyl, phenoxathiinyl, 2H-pyrrolyl, pyrrolyl, imidazolyl, pyrazolyl, isothiazolyl, isoxazolyl, oxazolyl, pyridinyl, pyrazinyl, pyrimidinyl, pyridazinyl, indolizinyl, isoindolyl, 3H-indolyl, indolyl, 1H-indazolyl, purinyl, 4H-quinolizinyl, isoquinolinyl, quinolinyl, phthalazinyl, naphthyridinyl, quinoxalinyl, quinazolinyl, cinnolinyl, pteridinyl, 4a H-carbazole, carbazole, beta-carbolinyl, phenanthridinyl, acridinyl, perimidinyl, phenanthrolinyl, phenazinyl, phenarsazinyl, phenothiazinyl, furazanyl, phenoxazinyl, pyrrolidinyl, pyrrolinyl, imidazolidinyl, imidazolinyl, pyrazolidinyl, pyrazolinyl, piperidinyl, piperazinyl, indolinyl, isoindolinyl, quinuclidinyl, morpholinyl or oxazolidinyl. Preferable heterocyclic groups include piperidino, morpholino, thiamorpholino, pyrrolidino, pyrazolino, pyrazolidino, pyrazoryl, piperazinyl, thienyl, oxazolyl, tetrazolyl, thiazolyl, imidazolyl, imidazolinyl, pyrazolyl, pyridyl, pyrimidinyl, pyrrolyl, pyrrolidinyl and quinolyl, each of which may be optionally substituted. More preferably, the basic nitrogen moiety is selected from the group consisting of carbazimidamide and optionally substituted piperidinyl, e.g. unsubstituted piperidinyl.

Typically, the HT group may comprise a group of the formula III:

Ar—C(O)-E-G-BN— (III)

(wherein Ar is a monocyclic or polycyclic aromatic or heteroaromatic;
E is selected from the group consisting of O and NH;
G is selected from the group consisting of C₁₋₆-alkyl, C₃₋₇-cycloalkyl, C₁₋₆-alkyl-C₃₋₇-cycloalkyl and C₃₋₇-cycloalkyl-C₁₋₆-alkyl; and
BN is a basic nitrogen moiety as herein defined;
or wherein G-BN together form a C₃₋₇-heteroalkyl, or a C₁₋₆-alkyl-C₃₋₇-heteroalkyl group).

Preferred HT groups are derivatives of piboserod having the formula IV:

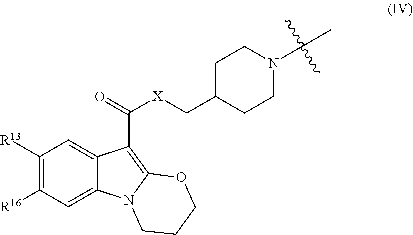

(wherein R¹³ is selected from the group consisting of H, halogen e.g. F, Cl, Br or I), NH₂ and C₁₋₆-alkyl; and
R¹⁶ is selected from the group consisting of H, halogen, OH, O—C₁₋₆-alkyl and C₁₋₆-alkyl).

Preferably, R¹³ and R¹⁶ are both H.

Other preferred HT groups are those of formula V:

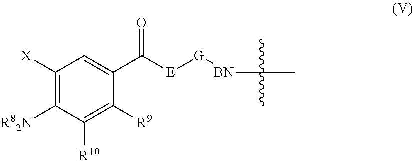

(wherein E is selected from the group consisting of O and NH;
G is selected from the group consisting of C₁₋₆-alkyl, C₃₋₇-cycloalkyl, C₁₋₆-alkyl-C₃₋₇-cycloalkyl and C₃₋₇-cycloalkyl-C₁₋₆-alkyl;
BN is a basic nitrogen moiety as herein defined;
or wherein G-BN together form a C₃₋₇-heteroalkyl, or a C₁₋₆-alkyl-C₃₋₇-heteroalkyl group;
X is a halogen;
R⁸ is independently selected from H and C₁₋₆-alkyl;
R⁹ and R¹⁰ are independently selected from the group consisting of H, O—C₁₋₆-alkyl, C₁₋₆-alkyl, a C₃₋₇-cycloalkyl, a heterocycloalkyl, a heteroaryl, or an aryl;
or wherein together R⁹ and R¹⁰ form a C₃₋₇-cycloalkyl, a heterocycloalkyl, a heteroaryl, or an aryl;
or wherein NR⁸₂ and R¹⁰ together form a heterocycloalkyl group).

Compounds of the formula V may be, for instance, amino benzamide derivatives or amino benzoates.

Specific examples of suitable HT groups include the pharmacophores of the 5-HT modulators described in WO 2007/007072, WO 2007/149929 and WO 2005/061483, the contents of each of which documents are incorporated in their entirety herein. Indole derivatives and compounds comprising three condensed ring systems, i.e. tricyclic derivatives are preferred. Especially preferred are oxazino-indole derivatives, such as those shown in Example 1. Where the HT group comprises an oxazino-indole derivative, group L may be a benzyl derivative, e.g. a —CH₂-p-phenylene.

Particularly preferred HT groups are those set forth in the following Examples, in particular the groups:

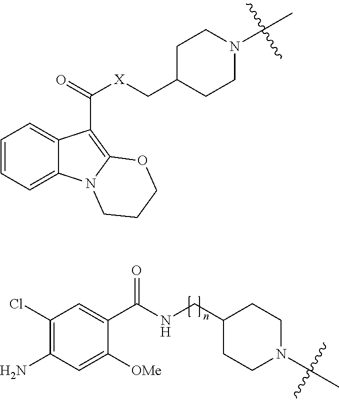

wherein X denotes O or NH, preferably NH, and n=0 or 1.

In general, unless it contains a sufficiently elongate rigid section, any linker (e.g. a group L) having three or more bonds in its backbone which separate the acid and the nitrogen but allow a free rotation which can bring the two closer together is likely to allow the two to approach too closely. Desirably, between the attachment site of the acid group and the attachment site of the pharmacophore, the linker contains no more than two, more preferably no more than one, backbone bond, rotation about which would cause the nitrogen and acid to come closer. Where the nitrogen atom is not the attachment site of the linker, the intervening portion of the pharmacophore desirably does not provide sufficient flexibility for the nitrogen and acid to approach too closely. Flexibility however may arise not just from rotations about bonds but from conformational changes and these too should be taken into account. Inter-group spacings may be assessed simply using conventional chemical modelling systems as bond angles and lengths may readily be calculated or determined from standard references.

The linking group of the invention must be rigid and/or sterically hindered such that the acidic moiety and the basic nitrogen atom of the 5-HT modulating moiety do not come into close contact. Bulky substituents include highly substituted alkyl groups such as C₁₋₁₂-alkyl groups substituted with one or more alkyl or aryl substituents, e.g. isopropyl and tertiary butyl substituents. Other hindered linkers include hydrophobic cyclic groups, e.g. C₃₋₈-cycloalkyl, C₄₋₈-cycloalkenyl and C₆₋₁₀-cycloaryl groups (e.g. benzyl). Rigid heterocyclic groups, e.g. 5- or 6-membered rings comprising 1 to 3 nitrogen, oxygen and/or sulphur atoms, may also be used as linkers according to the invention. Such heterocyclic groups can comprise unsaturated, saturated and polyunsaturated (e.g. aromatic) rings. Examples of heterocyclic linkers include piperidine, piperazine, pyrimidine, pyridine and benzothiazole groups.

Accordingly, in a preferred aspect of the invention, L comprises an optionally substituted mono- or bi-cyclic aryl or heteroaryl group; a linear C₁₋₆-alkyl group being substituted independently at each carbon atom by at least one optionally substituted C₁₋₆-alkyl, C₂₋₆-alkenyl, C₂₋₆-alkynyl, C₃₋₁₀-cycloalkyl, aryl, heteroaryl, nitrile, hydroxy, amide, chloride or iodide group; an optionally substituted C₃₋₁₀-cycloalkyl or C₄₋₁₀-cycloalkenyl group; or an optionally substituted polycyclic alkyl or alkenyl group, e.g. a group having a steroid backbone. Preferably, L is other than —CH₂-p-phenylene, —CO-p-phenylene and -p-phenylene.

Especially preferred groups L are benzyl, e.g. an ortho- or meta-benzyl group, which may be optionally substituted by one or more substituents including alcohol (hydroxy), amine, halide (e.g. F, Cl, Br or I), alkyl (e.g. C₁₋₆-alkyl), alkenyl (e.g. C₂₋₆-alkenyl) or alkynyl (e.g. C₂₋₆-alkynyl) substituents. Where L is a benzyl group, it is preferably not a para-benzyl group, especially preferably not an unsubstituted para-benzyl group.

In another preferred embodiment, L includes an optionally substituted, aromatic carbocyclic or aromatic heterocyclic group. Such groups possess an inherent rigidity which make them particularly well suited for use as rigid linkers according to the invention. Such linkers may, for example, comprise a group of the formula —(CH₂)ₙ—Ar′—(CR^{a}R^{b})ₘ— in which n is 0 or 1, preferably 1; Ar′ is an optionally substituted aryl ring or heteroaromatic ring; R^{a} and R^{b} are each independently H or, more preferably, optionally substituted C₁₋₆-alkyl (preferably C₁₋₄-alkyl, e.g. methyl); and m is 0 or 1, preferably 1. Preferably, the points of attachment of the HT moiety and the acid moiety (A) (or, where present, the —(CH₂)— and/or —(CR^{a}R^{b})— groups which in turn are linked to these moieties) on the aryl or heteroaromatic ring (Ar′) will be meta- or para- to one another, most preferably para. Particularly preferred groups L are those of formula —(CH₂)—Ar″—(CR^{a}R^{b})— in which Ar″ is optionally substituted phenyl, preferably unsubstituted phenyl. In such linker groups, the —(CH₂)— and —(CR^{a}R^{b})— groups are positioned either meta or para to one another. Where the phenyl group is para-substituted, the resulting linker and acid moieties may comprise a group of formula VI:

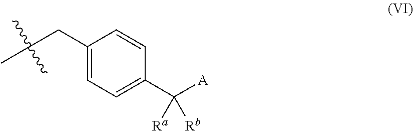

(wherein R^{a} and R^{b} are independently selected from H and optionally substituted C₁₋₆-alkyl; and A is an acid moiety as herein described). In a preferred embodiment of this aspect of the invention, at least one of R^{a} and R^{b} is C₁₋₆-alkyl (e.g. methyl) and especially preferably both R^{a} and R^{b} are C₁₋₆-alkyl groups (e.g. methyl).

In an alternative embodiment, L is an optionally substituted, optionally bridged C₄-C₁₀-cycloalkyl, preferably C₅-C₈-cycloalkyl, e.g. C₅-C₇-cycloalkyl, group. Optionally substituted, optionally bridged cyclopentyl and cyclohexyl groups are particularly preferred. In this embodiment, it is preferred that the points of attachment of the HT and A moieties on the cycloalkyl ring are not adjacent to one another. For example, where the cycloalkyl group is a cyclopentyl group, the HT and A moieties are preferably in a 1,3 relationship (i.e. meta to one another). This disposition of the groups on the cycloalkyl ring generally leads to a greater separation of the basic nitrogen and acid functionalities, thereby achieving the desired object of the present invention. However, it will be appreciated there will be certain embodiments in which adjacent positioning of the HT and A moieties, i.e. in a 1,2 relationship, will maintain the basic nitrogen and acid moieties sufficiently far apart to avoid interaction. For example, two adjacent groups in axial disposition on a cyclohexane ring may be maintained in an essentially rigid “para” disposition by virtue of the presence of one or more bulky substituents in equatorial positions on the cyclohexane ring. In this aspect of the invention, the cycloalkyl ring may be substituted with one or more groups independently selected from straight chained or branched C₁-C₆-alkyl, C₂-C₆-alkenyl and C₂-C₆-alkynyl groups, halogen (e.g. F, Cl, Br or I), oxo, hydroxy, C₁-C₆-alkoxy, cyano, amino, C₁-C₆-alkylamino and C₁-C₆-dialkylamino groups. Preferably the substituents are selected from groups which restrict the flexibility of the cycloalkyl ring, for example by steric or electronic interactions, such as one or more tert-butyl groups and/or halogen atoms. Up to three carbons of the cycloalkyl ring may be replaced by one or more heteroatoms selected from oxygen, sulphur and nitrogen. However, cycloalkyl groups without any heteroatom substitutions are preferred.

Bridging of a cycloalkyl group in the linker, L, introduces greater rigidity into the structure and so is a preferred aspect of the invention. A “bridging group” may represent a single bond which links two atoms of the cycloalkyl ring or may comprise one or more carbon, oxygen, sulphur or nitrogen atoms which bridge the cycloalkyl ring. Bridging groups consisting either of a bond or which comprise 1 or 2 atoms, especially 1 or 2 carbon atoms, are generally preferred. Bridging atoms may be independently substituted by one or more substituents as defined herein in respect of the cycloalkyl ring. Where one or more bridging groups are provided, linkage to the acid moiety (A) may either be via the main ring of the cycloalkyl group or, alternatively, via an atom which forms part of one of the bridging groups. Examples of suitable bridged cycloalkyl groups include bicyclo[2,2,1]heptane (norbornane), bicyclo[3,2,1]octane and adamantane. An especially preferred bridged cycloalkyl group is tricyclo[2,2,1,0^{2b}]heptane.

Another particularly preferred group L is biphenyl, especially methylene-para-biphenyl. In this embodiment, the group L-A is preferably a group of formula VII:

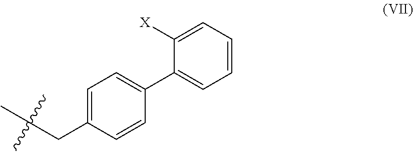

(wherein X is —C(O)OH, optionally substituted —C(O)O—C₁₋₆-alkyl or an optionally substituted 5-tetrazolyl group) or a prodrug form or salt thereof.

In a preferred embodiment of the invention, A denotes an acid moiety which is a protic acidic moiety having a labile proton. In a preferred embodiment, the labile proton, when in said acid moiety, is kept distanced from the basic nitrogen atom of the HT moiety by at least 0.6 nm by the linker moiety.

Preferred groups A include those described in WO 2005/061483, e.g. wherein A is selected from the group consisting of —C(O)—OR¹, —OP(O)OR²OR², —P(O)OR²OR², —SO₂OR², —SO₃H, —OSO₃H and —PO₃H; wherein R¹ and R² are independently selected from the group consisting of H, M (wherein M is a counterion), C₁₋₁₅-alkyl, C₃₋₈-cycloalkyl, aryl, and R^{1,2} wherein R^{1,2} is R′—O—C(O)R″, R′—O—C(O)—O—R″, R′—C(O)—O—R″, wherein R′ and R″ are independently selected from the group consisting of C₁₋₁₅-alkyl, C₃₋₈-cycloalkyl and aryl.

Particularly preferably, A denotes an oxyacid or a tetrazole group, or an ester or salt thereof, e.g. a carboxylic acid or an optionally substituted tetrazole group. By “oxyacid” is meant herein a group which in its protbnated form contains oxygen, hydrogen and an atom selected from C, S and P linked by a double bond to at least one oxygen or, less preferably, sulphur. Thus, for example, carboxyl (COOH) and its sulphur analogues (CSSH, CSOH and COSH) are covered, although carboxyl is preferred. The preferred S oxyacids are SO₃H and OSO₃H, while the preferred P oxyacids are OP(O)(OH)₂ and PO₃H.

In addition to the “rigid linker” aspect of the present invention, the inventors have also determined that certain 5-HT modulators may be beneficially provided with an acidic group having a renin-angiotensin system modulating activity. In particular, compounds having a 5-HT₄ modulatory activity and an angiotensin II receptor modulatory activity are described herein. Such dual-action modulators are new and form a further aspect of the invention.

Angiotensin II is a vasoactive peptide hormone produced from angiotensin I by the peptidase angiotensin converting enzyme (ACE). Drugs which interfere with the activity of this enzyme (so-called “ACE inhibitors”) can block the biosynthesis of angiotensin II and are widely used as cardiovascular drugs, e.g. as anti-hypertensives. Examples of such drugs include enalapril and captopril. Another class of cardiovascular drugs are the angiotensin II receptor antagonists, examples of which include the “sartans”, e.g. telmisartan, losartan, valsartan, candesartan and irbesartan.

In view of the effects of the 5-HT receptor modulators, especially 5-HT₄ receptor modulators, on the cardiovascular system, compounds which combine 5-HT modulatory activity with angiotensin receptor modulatory function are uniquely placed for use in the treatment of cardiovascular diseases, especially congestive heart failure.

According to this aspect, the present invention provides compounds of formula Ib:

HT-L_{b}-A_{b} (Ib)

as well as the prodrugs and salts thereof, wherein HT is as hereinbefore defined and L_{b} is absent or is any linker which enables the pharmacophores of HT (5-HT receptor modulation) and A_{b} (renin-angiotensin system modulating activity) to function.

L_{b} is preferably a rigid linker L as hereinbefore defined, but may also be a non-rigid linker as described in WO 2007/007072, WO 2007/149929 and WO 2005/061483. Examples of linkers L_{b} according to the invention include, in addition to those defined above for L, straight chain or branched, optionally substituted C₁₋₁₀-alkyl, optionally substituted C₂₋₁₀-alkenyl, optionally substituted C₂₋₁₀-alkynyl, C₁₋₁₀-alkylamine, C₁₋₁₀-alkoxy, C₂₋₁₀-alkenyloxy, C₂₋₁₀-alkynyloxy, C₁₋₁₀-alkoxycarbonyl, C₂₋₁₀-alkenyloxycarbonyl and C₂₋₁₀-alkynyloxycarbonyl groups.

In a preferred embodiment, A_{b} denotes the pharmacophore of an ACE inhibitor or an angiotensin II receptor antagonist. Preferably, A_{b} denotes the pharmacophore of an angiotensin II receptor antagonist. The definition and scope of the term “pharmacophore” in this context would be clear to the person skilled in the art.

A_{b} preferably denotes an acidic pharmacophore, particularly preferably denoting a pharmacophore comprising a biphenyl, especially a methylene-para-biphenyl group. Groups of formula VII as herein defined wherein X is —C(O)OH, optionally substituted —C(O)O—C₁₋₆-alkyl or an optionally substituted 5-tetrazolyl group, or a prodrug form or salt thereof, are especially preferred. Preferred groups of formula VII are those wherein X is —C(O)OH, —C(O)OCH₃ or optionally substituted tetrazole, e.g. N-trityl-tetrazole.

The compounds of the invention are 5-HT receptor modulators, typically 5-HT₄ receptor modulators. The compounds may be 5-HT (e.g. 5-HT₄) agonists or antagonists. Alternatively, these may be partial agonists.

By “5-HT receptor modulator” is meant any compound having 5-HT receptor modulatory activity described herein. Examples of such compounds include those of formula I and lb. Especially preferred 5-HT receptor modulators include compounds 1-9, 11-15, 17-21, 22a-f and 23-30 as described in the Examples.

The conditions which may be treated using the compounds herein described include any which may be responsive to 5-HT receptor agonism or antagonism. Such conditions may be associated, for example, with diseases of the urinary system, the gastrointestinal system, or the cardiovascular system. Examples of particular conditions which may be treated using the compounds of the invention include gastroesophageal reflux, diarrhoea, abdominal cramps, dyspepsia, gastroparesis, constipation, post-operative ileus, intestinal pseudo-obstruction, irritable bowel syndrome, bladder diseases (e.g. hyperactive bladder, etc.), hypertension, pulmonary hypertension, portal hypertension, cardia hypertrophy and cardiac valve disease.

Viewed from a further aspect the invention provides a pharmaceutical composition comprising the 5-HT receptor modulator, e.g. a compound of formula I or Ib, or a physiologically tolerable prodrug form or salt thereof, together with at least one pharmaceutical carrier or excipient.

The carriers or excipients used in the compositions may be any of the materials commonly used in pharmaceutical compositions, e.g. solvents (such as water), pH modifiers, viscosity modifiers, fillers, diluents, binders, aromas, skin penetration enhancers, antioxidants and other preservatives, etc. The choice will depend on the dosage administration route and form. Typically, the compositions will be sterile.

The compositions of the invention may be in any convenient dosage administration form, e.g. solutions, dispersions, suspensions, syrups, tablets, coated tablets, powders, sprays, suppositories, etc. Solutions, dispersions and tablets are preferred. These may be prepared in conventional fashion.

The administration route for the compounds and compositions of the invention may be enteral, e.g. oral, rectal or by tube, nasal, sub-lingual, by injection or infusion.

Viewed from another aspect the invention provides a 5-HT receptor modulator as herein described, e.g. compound of formula I, or a physiologically tolerable prodrug form or salt thereof for use in medicine.

Viewed from a still further aspect the invention provides the use of a 5-HT receptor modulator as herein described, such as a compound of formula I, or a physiologically tolerable prodrug form or salt thereof for use in the treatment of a 5-HT associated condition, e.g. for the manufacture of a medicament for use in a method of treatment of a 5-HT associated condition. Examples of 5-HT associated condition are'known to the skilled person and include diseases of the cardiovascular system, diseases of the, gastrointestinal system and diseases of the urinary system, especially cardiac failure.

Viewed from another aspect the invention provides a method of treatment of diseases of the cardiovascular system, the gastrointestinal system and the urinary system, said method comprising the step of administering a therapeutically effective amount of a 5-HT receptor modulator as herein described. In a preferred embodiment, the invention provides a method of treatment of cardiac failure.

Diseases of the urinary system which may be treated particularly readily using the compounds of the invention are diseases of the lower urinary tract.

In the methods of the invention, the compounds may typically be administered at dosages of from about 0.1 mg to about 200 mg in single or divided doses. Preferably a daily dose should be between about 1 mg to about 100 mg, more preferably between about 2 mg and 75 mg. It may be necessary to use dosages outside these ranges in some cases as will be apparent to those skilled in the art.

The synthesis of the 5-HT modulators of the invention may be performed using synthetic methodology well known in organic chemistry. Typical methods include alkylation of the basic nitrogen of the HT moiety with an alkylating agent comprising the acidic group or prodrug of the acidic group. For example, the HT moiety may be alkylated with a bromomethyl biphenyl derivative comprising a protected acid group. An alternative method would involve alkylation of nitrogen using an alkylating agent which comprises an aromatic cyano group, followed by reaction with an azide to yield a tetrazole. The inventors also contemplate building a 5-HT pharmacophore on to an acidic hydrophobic scaffold using known methodology.

The preparation of representative compounds of the invention is illustrated by way of the following non-limiting examples:

### EXAMPLE 1

### Compounds 1-8

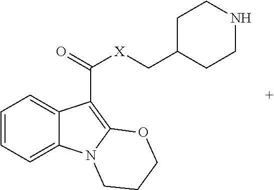

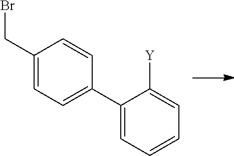

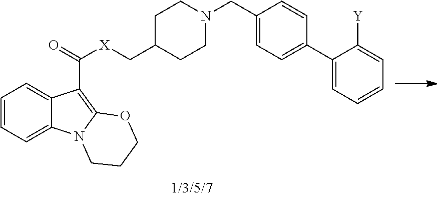

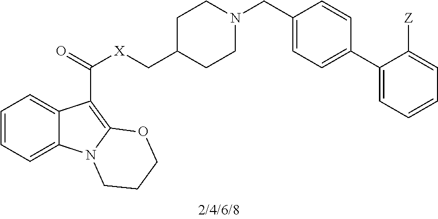

| Com- | | | | Molecular |
|---|---|---|---|---|
| pound | X | Y | Z | formula |
| 1 | NH | 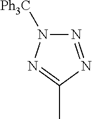 | | C₅₁H₄₇N₇O₂ |
| 2 | NH | | 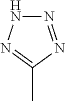 | C₃₂H₃₃N₇O₂ |
| 3 | O | 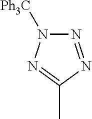 | | C₅₁H₄₆N6O₃ |
| 4 | O | | 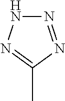 | C₃₂H₃₂N₆O₃ |
| 5 | NH | 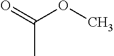 | | C₃₃H₃₅N₃O₄ |
| 6 | NH | | 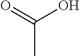 | C₃₂H₃₃N₃O₄ |
| 7 | O | 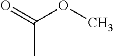 | | C₃₃H₃₄N₂O₅ |
| 8 | O | | 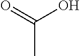 | C₃₂H₃₂N₂O₅ |

### Compound 1

N-Trityl-5-[(4′-bromomethyl)-biphenyl-2-yl]tetrazole (1.05 g, 3.0 mmol) was added to a stirred suspension of N-(4-piperidylmethyl) 3,4-dihydro-2H-[1,3]oxazino[3,2-a]indole-10-carboxamide (1.05 g, 3.0 mmol) and K₂CO₃ (1.65 g, 12.0 mmol) in acetone (30 ml) and heated to reflux for 24 h. The mixture was cooled to room temperature and filtered. The filtrate was evaporated in vacuo and the residue added CH₂Cl₂ (50 ml) and washed with H₂0 (3×25 ml). The organic layer was dried over Na₂SO₄, filtered and evaporated in vacuo. The residue was separated with flash chromatography (CH₂Cl₂/MeOH (9:1) to leave the intermediate 1 as a yellow oil (0.60 g, 25.3%). ¹H NMR (CDCl₃): δ 8.32 (d, 1H), 7.87 (d, 1H), 7.41-7.17 (m, 16H), 7.10-7.08 (m, 6H), 6.89-6.84 (m, 6H), 6.54 (t, 1H), 4.51 (t, 2H), 4.06 (t, 2H), 3.40 (br s, 2H), 3.30 (t, 2H), 2.90-2.82 (m, 2H), 2.37-2.26 (p, 2H), 2.02-1.88 (m, 2H) 1.71-1.50 (m, 2H), 1.35-1.24 (m, 2H). MS (ES): 790.1 [M+H]⁺

### Compound 2

Compound 1 (0.50 g, 0.63 mmol) was stirred in a mixture of CH₂Cl₂/TFA/H₂O (97:2:1, 25 ml) at room temperature overnight and evaporated in vacuo. The residue was separated with flash chromatography (CH₂Cl₂, MeOH, 9:1) to leave the free tetrazol compound 2 as a white solid (0.31, 90.1%). ¹H NMR (DMSO-_{d6}): δ 11.07 (br s, 1H), 8.61 (br s, 1H), 8.01 (d, 1H), 7.87-7.04 (m, 12H), 6.91 (t, 1H), 4.49 (t, 2H), 4.27 (br s, 2H), 4.13 (t, 2H), 3.34-3.24 (m, 2H), 2.95-2.85 (m, 2H), 2.28-2.23 (m, 2H), 1.89-1.60 (m, 5H). MS (ES): 548.1 [M+H]⁺

### Compound 3

Following the procedure outlined for compound 1, N-(4-piperidylmethyl) 3,4-dihydro-2H-[1,3]oxazino[3,2-a]indole-10-carboxylate (0.31 g, 0.88 mmol) and N-Trityl-5-[(4′-bromomethyl)-biphenyl-2-yl]tetrazole (0.49 g, 0.88 mmol) was converted to intermediate 3 as a white solid (0.34 g, 48.7%). ¹H NMR (CDCl₃): δ 7.93-7.88 (m, 2H), 7.29-7.06 (m, 18H), 6.89-6.84 (m, 6H), 4.51 (t, 2H), 4.17-4.05 (m, 4H), 3.38 (br s, 2H), 2.85-2.80 (m, 2H), 2.37-2.26 (p, 2H), 2.02-1.72 (m, 6H) 1.71-1.50 (m, 2H). MS (ES): 791.1 [M+H]⁺

### Compound 4

Following the procedure outlined for compound 2, the trityl group of compound 3 (0.24 g, 0.30 mmol) was cleaved to leave the free tetrazol compound 4 as a white solid (0.14 g, 85.3%). ¹H NMR (DMSO-_{d6}): δ 9.70 (br s, 1H), 7.75 (d, 1H), 7.69-7.67 (m, 2H), 7.62-7.57 (m, 2H), 7.44 (d, 2H), 7.31 (d, 2H), 7.19-7.08 (m, 4H), 4.49 (t, 2H), 4.27 (br s, 2H), 4.13-3.94 (m, 4H), 3.40-3.37 (m, 2H), 2.96 (t, 2H), 2.28-2.23 (m, 2H), 2.02.1.90 (m, 3H), 1.52-1.40 (m, 2H). MS (ES): 549.1 [M+H]⁺

### Compound 5

Following the procedure outlined for compound 1, N-(4-piperidylmethyl) 3,4-dihydro-2H-[1,3]oxazino[3,2-a]indole-10-carboxamide (1.05 g, 3.0 mmol) and 4-bromomethyl-(1,1-biphenyl)-2-carboxylic acid methyl ester (0.91 g, 3.0 mmol) was converted to compound 5 as a white solid (0.65 g, 40.3%). ¹H NMR (CDCl₃): δ 8.31 (d, 1H), 7.82 (d, 1H), 7.53-7.10 (m, 16H), 7.10-7.08 (m, 6H), 6.89-6.84 (m, 6H), 6.54 (t, 1H), 4.51 (t, 2H), 4.06 (t, 2H), 3.40 (br s, 2H), 3.30 (t, 2H), 2.90-2.82 (m, 2H), 2.37-2.26 (p, 2H), 2.02-1.88 (m,2H) 1.71-1.50 (m, 2H), 1.35-1.24 (m, 2H). MS (ES): 538.1 [M+H]⁺

### Compound 6

Compound 5 (0.40 g, 0.74 mmol) was stirred in a mixture of 2 M aqueous NaOH solution (1 ml) and MeOH (4 ml) and heated to reflux for 12 h, cooled to room temperature and evaporated in vacuo. The residue was redissolved in H₂O (5 ml) and the solution acidified to pH 2 with 2 M aqueous HCl. The free carboxylic acid 6 precipitated out of the solution, the precipitate filtered off and the residue recrystallized from acetone (0.20 g, 51.6%). ¹H NMR (DMSO-_{d6}): δ 8.04 (d, 1H), 7.71 (d, 1H), 7.58-7.34 (m, 7H), 7.27 (d, 1H), 7.08-7.03 (m, 2H), 6.87 (t, 1H), 4.54 (t, 2H), 4.10 (t, 2H), 3.96 (br s, 2H), 3.20 (t, 2H), 3.10 (d, 2H), 2.52-2.48 (m, 2H), 2.30-2.25 (m, 2H), 1.77-1.70 (m, 3H), 1.50-1.42 (m, 2H). MS (ES): 524.1 [M+H]⁺

### Compound 7

Following the procedure outlined for compound 1, N-(4-piperidylmethyl) 3,4-dihydro-2H-[1,3]oxazino[3,2-a]indole-10-carboxylate (1.05 g, 3.0 mmol) and 4-bromomethyl-(1,1-biphenyl)-2-carboxylic acid methyl ester (0.91 g, 3.0 mmol) was converted to compound 7 as a white solid (0.86 g, 53.0%). ¹H NMR (CDCl₃): δ 7.95 (d, 1H), 7.77 (d, 1H), 7.49-7.10 (m, 10H), 4.50 (t, 2H), 4.17 (d, 2H), 4.06 (t, 2H), 3.59 (s, 2H), 3.52 (s, 2H), 2.95-2.84 (m, 2H), 2.37-2.27 (m, 2H), 2.06-1.79 (m, 5H) 1.71-1.50 (m, 2H), 1.49-1.41 (m, 2H). MS (ES): 539.1 [M+H]⁺

### Compound 8

Following the procedure outlined for compound 6, the compound from example 7 (1.51 g, 2.80 mmol) was converted to the free acid 8 as a white solid (1.04, 71.5%). ¹H NMR (DMSO-_{d6}): δ 7.81 (d, 1H), 7.60 (d, 1H), 7.42-7.28 (m, 8H), 7.13-7.07 (m, 2H), 4.49 (t, 2H), 4.10 (t, 2H), 4.04 (d, 2H), 3.66 (s, 2H), 2.84 (d, 2H), 2.28-2.22 (m, 2H), 2.01 (t, 2H), 1.74-1.70 (m, 3H), 1.37-1.3.1 (m, 2H). MS (ES): 525.1 [M+H]⁺

### EXAMPLE 2

### Compounds 9-20

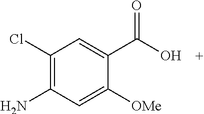

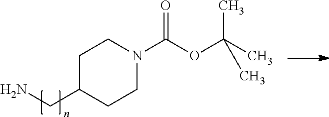

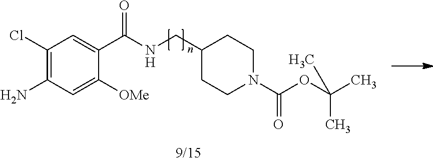

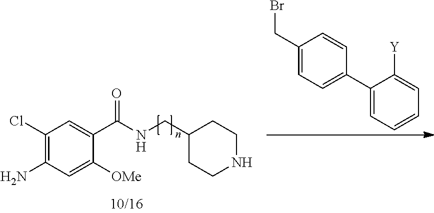

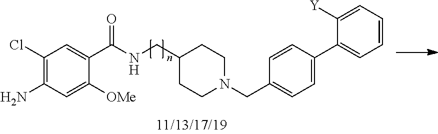

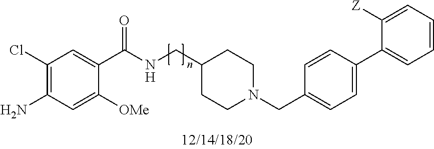

| | | | | Molecular |
|---|---|---|---|---|
| Compound | n | Y | Z | formula |
| 9 | 0 | // | // | // |
| 10 | 0 | // | // | // |
| 11 | 0 | 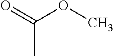 | | C₂₈H₃₀ClN₃O₄ |
| 12 | 0 | | 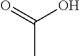 | C₂₇H₂₈ClN₃O₄ |
| 13 | 0 | 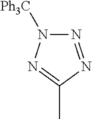 | | C₄₆H₄₂ClN₇O₂ |
| 14 | 0 | | 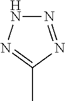 | C₂₇H₂₈ClN₇O₂ |
| 15 | 1 | // | // | // |
| 16 | 1 | // | // | // |
| 17 | 1 | 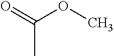 | | C₂₉H₃₂ClN₃O₄ |
| 18 | 1 | | 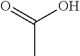 | C₂₈H₃₀ClN₃O₄ |
| 19 | 1 | 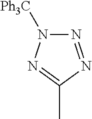 | | C₄₇H₄₄ClN₇O₂ |
| 20 | 1 | | 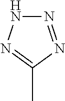 | C₂₈H₃₀ClN₇O₂ |

### Compound 9

A mixture of 4-amino-1-Boc-piperidine (2.0g, 10.0 mmol), 4-amino-5-chloro-2-methoxybenzoic acid (2.01 g, :10.0 mmol) and NEt₃ (1.01 g, 10.0 mmol) in DMF (40 ml) were added 1-ethyl-3-[3-(dimethylamino)propyl]carbodiimide hydrochloride (EDC) (1.91 g, 10.0 mmol) and 1-hydroxybenzotriazole (HOBT) (1.35 g, 10.0 mmol) at 0° C. The reaction mixture was stirred to room temperature overnight and concentrated in vacuo. The resulting residue was redissolved in CH₂Cl₂ (100 ml) and extracted with aqueous K₂CO₃ (3×50 ml). The organic layer was dried over Na₂SO₄, filtered and evaporated in vacuo to leave the intermediate 4-Amino-5-chloro-2-methoxy-N-(1-Boc-4-piperidyl)benzamide 9 as a white solid. (3.93, 98.4%) ¹H NMR (CDCl₃): δ 8.06 (s, 1H), 7.60 (d, 1H), 6.26 (s, 1H), 4.40 (s, 2H), 4.10-4.08 (m, 1H), 3.94 (d, 2H), 3.85 (s, 3H), 2.96 (t, 2H), 1.98-1.93 (m, 2H), 1.43 (s, 9H), 1.40-1.36 (m, 2H).

### Compound 10

A solution of 4-amino-5-chloro-2-methoxy-N-(1-Boc-4-piperidyl)benzamide (3.93 g, 9.84 mmol) in dioxane (25 ml) was cooled to 0° C. and added 4M HCl in dioxane (2.0 ml) and stirred to room temperature for 4 h. The mixture was evaporated in vacuo, added MeOH (20 ml) and heated under reflux for 1 h. The reaction mixture was evaporated in vacuo and the residue recrystallized from EtOH to leave the intermediate 4-Amino-5-chloro-2-methoxy-N-(4-piperidyl) benzamide hydrochloride 10 as a white crystalline solid (2.01 g, 62.8%). ¹H NMR (DMSO-d₆): δ 9.19-9.13 (m, 2H), 7.76 (d, 1H), 7.60 (s, 1H), 6.52 (s, 1H), 6.16 (br s, 3H), 4.99 (br s, 2H), 3.81 (s, 3H), 3.23-3.19 (m, 2H), 2.99-2.95 (m, 2H), 2.01-1.97 (m, 2H), 1.78-1.67 (m, 2H).

### Compound 11

Following the procedure outlined for compound 5, 4-amino-5-chloro-2-methoxy-N-(4-piperidyl)benzamide hydrochloride (1.60 g, 5.0 mmol) and 4-bromomethyl-(1,1-biphenyl)-2-carboxylic acid methyl ester (1.52 g, 5.0 mmol) was converted to compound 11 as a yellow solid (0.59 g, 23.2%). ¹H NMR (CDCl₃): δ 8.07 (s, 1H), 7.81-7.76 (m, 1H), 7.62 (d, 1H), 7.54-7.34 (m, 5H), 7.31-7.22 (m, 3H), 6.26 (s, 1H), 4.33 (s, 2H), 4.11-3.97 (m, 1H), 3.84 (s, 3H), 3.60 (s, 3H), 3.54 (s, 2H), 2.85-2.74 (m, 2H), 2.28-2.18 (m, 2H), 2.02-1.97 (m, 2H), 1.63-1.48 (m, 4H). MS (ES): 508.2 [M+H]⁺

### Compound 12

Following the procedure outlined for compound 6, the compound from example 11 (0.51 g, 1.00 mmol) was converted to the free acid 12 as a white solid (0.34 g, 69.3%). ¹H NMR (DMSO-d₆): δ 7.68-7.54 (m, 3H), 7.52-7.25 (m, 7H), 6.49 (s, 1H), 5.94 (s, 2H), 3.83 (s, 3H), 3.60 (s, 2H), 2.75-2.82 (m, 2H), 2.30-2.20 (m, 2H), 1.90-1.80 (m, 2H), 1.67-1.55 (m, 2H). MS (ES): 494.2 [M+H]⁺

### Compound 13

Following the procedure outlined for compound 1, 4-Amino-5-chloro-2-methoxy-N-(4-piperidyl)benzamide hydrochloride (0.80 g, 2.50 mmol) and N-trityl-5-[(4′-bromomethyl)-biphenyl-2-yl]tetrazole (1.39 g, 2.5 mmol) was converted to intermediate 13 as a white solid (98 mg, 6.4%). ¹H NMR (CDCl₃): δ 8.07 (s, 1H), 7.85-7.80 (m, 1H), 7.6 (d, 1H), 7.51-7.30 (m, 4H), 7.30-7.15 (m, 7H), 7.06 (s, 4H), 6.89-6.84 (m, 6H), 6.26 (s, 1H), 4.34 (s, 2H), 4.12-4.04 (m, 1H),3.84 (s, 3H), 3.37 (s, 2H), 2.77-2.66 (m, 2H), 2.17-2.07 (m, 2H), 1.94-1.90 (m, 2H), 1.61-1.43 (m, 5H).

### Compound 14

Following the procedure outlined for compound 2, the compound from example 13 (1.05 g, 1.38 mmol) was converted to free tetrazaol 14 as a white solid (0.43 g, 56.3%). ¹H NMR (DMSO-_{d6}): δ10.8 (br s, 1H), 7.71-7.45 (m, 7H), 7.16-7.12 (m, 2H), 6.46 (s, 1H), 4.34-4.21 (m, 2H), 3.95-3.80 (m, 1H), 3.77 (s, 3H), 3.31-3.20 (m, 2H), 3.10-2.97 (m, 2H), 2.04-1.80 (m, 4H). MS (ES): 518.2 [M+H]⁺

### Compound 15

Following the procedure outlined for compound 9, 4-amino-5-chloro-2-methoxybenzoic acid (2.01 g, 10.0 mmol) and 1-Boc-4-(aminomethyl)piperidine was converted to 4-amino-5-chloro-2-methoxy-N-(1-Boc-4-methylpiperidyl)benzamide 15 as a white solid (3.90 g. 94.8%). ¹H NMR (CDCl₃): δ 8.11 (s, 1H), 7.28 (s, 5H), 6.31 (s, 1H), 4.12 (d, 2H), 3.91 (s, 3H), 3.33 (d, 2H), 2.70 (t, 2H), 1.81-1.66 (m, 3H), 1.46 (s, 9H), 1.26-1.12 (m, 2H).

### Compound 16

Following the procedure outlined for compound 10, 4-amino-5-chloro-2-methoxy-N-(1-Boc-4-methylpiperidyl)benzamide (3.90 g, 9.46 mmol) was converted to intermediate 4-amino-5-chloro-2-methoxy-N-(4-methylpiperidyl)benzamide hydrochloride 16 as a white solid (2.35 g, 74.6%). ¹H NMR (DMSO-d₆): δ 9.24 (d, 1H), 9.03-8.96 (m, 1H), 7.99 (t, 1H), 7.65 (s, 1H), 6.55 (s, 1H), 3.82 (s, 3H), 3.22-3.14 (m, 4H), 2.83-2.72 (m, 2H), 1.79-1.71 (m, 3H), 1.45-1.33 (m, 2H).

### Compound 17

Following the procedure outlined for compound 5, 4-amino-5-chloro-2-methoxy-N-(4-methylpiperidyl)benzamide hydrochloride (1.67 g, 5.0 mmol) and 4-bromomethyl-(1,1-biphenyl)-2-carboxylic acid methyl ester (1.52 g, 5.0 mmol) was converted to compound 17 as a white solid (0.67 g, 25.6%). ¹H NMR (CDCl₃): δ 8.08 (s, 1H), 7.80-7.72 (m, 2H), 7.53-7.20 (m, 8H), 6.26 (s, 1H), 4.35 (s, 2H), 3.86 (s, 3H), 3.60 (s, 3H), 3.51 (s, 2H), 3.31 (t, 2H), 2.94-2.85 (m, 2H), 2.02-1.92 (m, 2H), 1.73-1.56 (m, 4H), 1.43-1.20 (m, 2H).

### Compound 18

Following the procedure outlined for compound 6, compound 17 (0.52 g, 1.00 mmol) was converted to the free acid 18 as a white solid (0.29 g, 57.1%). MS (ES): 508.1 [M+H]⁺

### Compound 19

Following the procedure outlined for compound 1, 4-Amino-5-chloro-2-methoxy-N-(4-methylpiperidyl)benzamide hydrochloride (1.67 g, 5.00 mmol) and N-trityl-5-[(4′-bromomethyl)-biphenyl-2-yl]tetrazole (2.78 g, 5.0 mmol) was converted to intermediate 19 as a white solid (1.05 g, 29.1%). ¹H NMR (CDCl₃): δ 8.07 (s, 1H), 7.98 (s, 1H), 8.00-7.98 (m, 1H), 7.90-7.85 (m, 1H), 7.77 (t, 1H), 7.44-7.16 (m, 11H), 7.04 (s, 4H), 6.89-6.84 (s, 6H), 6.27 (s, 1H), 4.24 (s, 2H), 3.82 (s, 3H), 3.34 (s, 2H), 3.28 (t, 2H), 2.85-2.77 (m, 2H), 1.92-1.81 (m, 3H), 1.64-1.54 (m, 3H), 1.35-1.23 (m, 2H).

### Compound 20

Following the procedure outlined for compound 2, compound 19 (1.03 g, 1.33 mmol) was converted to the free tetrazol compound 20 as a white solid (0.49 g, 64.9%). ¹H NMR (DMSO-_{d6}): δ10.59 (br s, 1H), 7.95 (t, 1H), 7.80-7.49 (m, 6H), 7.15-7.11 (m, 2H), 6.44 (s, 1H), 4.29-4.18 (m, 2H), 3.78 (s, 3H), 3.47-3.23 (m, 2H), 3.20-3.01 (m, 3H), 2.90-2.69 (m, 2H), 1.80-1.43 (m, 4H). MS (ES): 532.2 [M+H]⁺

### EXAMPLE 3

### Prodrugs of Compound 4 and 8

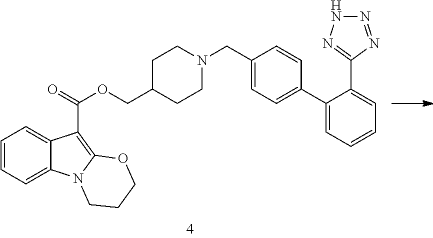

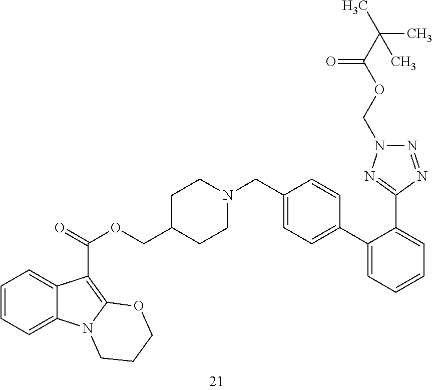

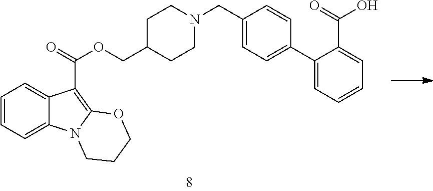

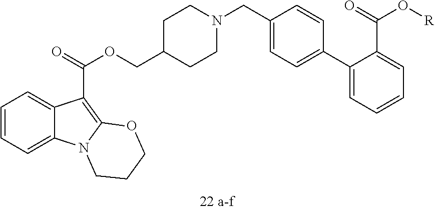

| | | | Molecular |
|---|---|---|---|
| Compound | X | R | formula |
| 21 | O | 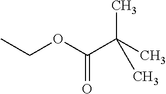 | C₃₈H₄₂N₆O₅ |
| 22a | O | 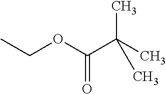 | C₃₈H₄₂N₂O₇ |
| 22b | O | 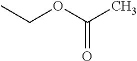 | C₃₅H₃₆N₂O₇ |
| 22c | O | 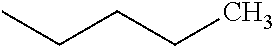 | C₃₆H₄₀N₂O₅ |
| 22d | O | 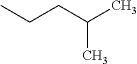 | C₃₇H₄₂N₂O₅ CH₃SO₃H |
| 22e | O | 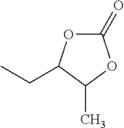 | C₃₇H₃₆N₂O₈ |
| 22f | O | 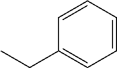 | C₃₉H₃₈N₂O₅ CH₃SO₃H |

### Compound 21

Chloromethyl pivalate (0.054 g, 0.36 mmol) was added to a mixture of tetrazole compound 4 (0.158 g, 0.29 mmol) and caesium carbonate (0.094 g, 0.29 mmol) in DMF (1.0 ml). The mixture was heated to 60° C. for 12 hours, cooled to room temperature and evaporated in vacuo. The residue was separated with flash chromatography (SiO₂, CH₂Cl₂:MeOH 9:1) to leave the prodrug 21 as a white solid (0.030 g, 15.6%). ¹H NMR (CDCl₃): δ 7.97 (d, 1H), 7.80 (d, 1H), 7.55-7.45 (m, 3H), 7.26-7.10 (m, 7H), 6.36 (s, 2H), 4.54 (t, 2H), 4.20 (d, 2H), 4.11 (t, 2H), 3.49 (br s, 2H), 2.95-2.88 (m, 2H), 2.37-2.32 (m, 2H), 2.04-1.82 (m, 5H), 1.60-1.45 (m, 2H), 1.17 (s, 9H)

### Compound 22a

Following the procedure outlined for compound 21, compound 8 (0.20 g, 0.38 mmol) and chloromethyl pivalate (0.057 g, 0.38 mmol) was converted to the prodrug 22a as a white solid. ¹H NMR (CDCl₃): δ 7.94 (d, 1H), 7.82 (d, 1H), 7.53-7.10 (m, 10H), 5.74 (s, 2H), 4.51 (t, 2H), 4.18 (d, 2H), 4.08 (t, 2H), 3.55 (br s, 2H), 2.99-2-92 (m, 2H), 2.37-2.25 (m, 2H), 2.10-1.86 (m, 5H), 1.49-1.44 (m, 2H), 1.16 (s, 9H).

### Compound 22b

Following the procedure outlined for compound 21, compound 8 (0.20 g, 0.38 mmol) and chloromethyl acetate (0.041 g, 0.38 mmol) was converted to the prodrug 22b as a white solid (0.040 g, 16.7%). ¹H NMR (CDCl₃): δ 7.94 (d, 1H), 7.82 (d, 1H), 7.53-7.11’(m, 10H), 5.71 (s, 2H), 4.52 (t, 2H), 4.18 (d, 2H), 4.09 (t, 2H), 3.53 (br s, 2H), 2.97-2.93 (m, 2H), 2.38-2.26 (m, 2H), 2.07-1.97 (m, 5H), -1.86-1.80 (m, 3H), 1.53-1.41 (m, 2H).

### Compound 22c

Following the procedure outlined for compound 21, compound 8 (0.10 g, 0.19 mmol) and n-butyl bromide (0.035 g, 0.19 mmol) was converted to the prodrug 22c as a white solid (0.028 g, 25.4%). ¹H NMR (CDCl₃): δ 7.94 (d, 1H), 7.80 (d, 1H), 7.52-7.11 (m, 10H), 4.50 (t, 2H), 4.18 (d, 2H), 4.10-3.98 (m, 4H), 3.52 (br s, 2H), 2.97-2.92 (m, 2H), 2.36-2.25 (m, 2H), 2.07-1.96 (m, 2H), 1.85-1.80 (m, 3H), 1.53-1.25 (m, 4H), 1.14-1.02 (m, 2H), 0.77 (t, 2H).

### Compound 22d

Following the procedure outlined for compound 21, compound 8 (0.20 g, 0.38 mmol) and 3-methyl-1-bromobutane (0.057 g, 0.38 mmol) was converted to the prodrug 22d as a white solid (0.049 g, 21.7%). The corresponding mesylate salt was prepared. ¹H NMR (DMSO-_{d6}): δ 9.46 (br s, 1H), 7.76-7.74 (m, 2H), 7.63-7.57 (m, 4H), 7.54-7.36 (m, 3H), 7.29 (d, 1H), 7.12-7.10 (m, 2H), 4.51-4.47 (m, 2H), 4.35-4.33 (m, 2H), 4.11-4.07 (m, 4H), 4.01 (t, 2H), 3.51-3.47 (m, 2H), 3.33-3.30 (m, 1H), 3.14-3.11 (m, 2H), 2.35 (s, 3H), 2.27-2.23 (m, 2H), 2.01-1.98 (m, 2H), 1.55-1.52 (m, 2H), 1.31-1.29 (m, 1H), 1.25-1.22 (m, 2H), 0.74 (d, 6H).

### Compound 22e

Following the procedure outlined for compound 21, compound 8 (0.20 g, 0.38 mmol) and 4-chloromethyl-5-methyl-1,3-dioxol-2-one (0.056 g, 0.38 mmol)) was converted to the prodrug 22e as a white solid (0.039 g, 16.2%). ¹H NMR (CDCl₃): δ 7.95 (d, 1H), 7.78 (d, 1H), 7.53-7.11 (m, 10H), 4.78 (s, 2H), 4.52 (t, 2H), 4.18 (d, 2H), 4.08 (t, 2H), 3.57 (br s, 2H), 2.94-2.90 (m, 2H), 2.35-2.29 (m, 2H), 2.07-2.04 (m, 5H), 1.86-1.81 (m, 3H), 1.53-1.39 (m, 3H).

### Compound 22 f

Following the procedure outlined for compound 21, compound 8 (0.20 g, 0.38 mmol) and benzyl bromide (0.065 g, 0.38 mmol) was converted to the prodrug 22f as a white solid (0.044 g, 21.0%). ¹H NMR (CDCl₃): δ 10.09 (br s, 1H), 7.92 (d, 2H), 7.45-7.43 (m, 1H), 7.37-7.28 (m, 6H), 7.17-7.12 (m, 8H), 5.12 (s, 2H), 4.55 (t, 2H), 4.28-4.24 (m, 4H), 4.10 (t, 2H), 3.65-3.61 (m, 2H), 2.95.(s, 3H), 2.71-2.69 (m, 2H), 2.36-2.33 (m, 2H), 2.00-1.95 (m, 4H). The corresponding hydrochloride salt was prepared.

### EXAMPLE 4

### Compounds 23-30

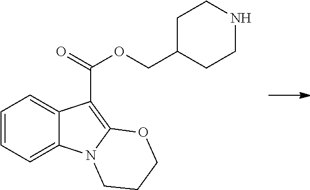

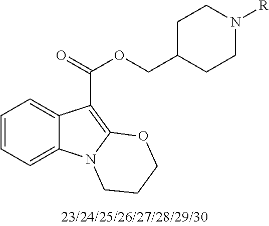

| Com- | | Molecular |
|---|---|---|
| pound | R | formula |
| 23 | 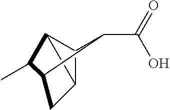 | C₂₆H₃₀N₂O₅ |
| 24 | 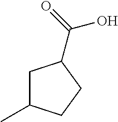 | C₂₄H₃₀N₂O₅ |
| 25 | 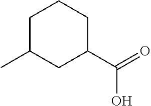 | C₂₅H₃₂N₂O₅ |
| 26 | 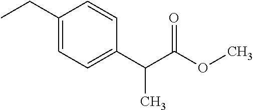 | C₂₉H₃₄N₂O₅ |
| 27 | 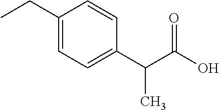 | C₂₈H₃₂N₂O₅ |
| 28 | 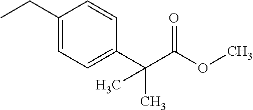 | C₃₀H₃₆N₂O₅ |
| 29 | 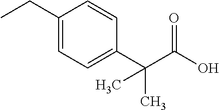 | C₂₉H₃₄N₂O₅ |
| 30 | 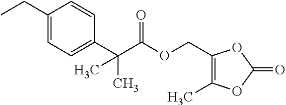 | C₃₄H₃₈N₂O₈ |

### Compound 23

Anti-3-oxotricyclo[2,2,1,0^{2b}]-heptane-7-carboxylic acid (0.12 g, 0.76 mmol) was added to a suspension of N-(4-piperidylmethyl)-3,4-dihydro-2H-[1,3]oxazino[3,2-a]indole-10-carboxylate (0.20 g, 0.64 mmol), NaCNBH₃ (1.65 g, 12.0 mmol) and molecular sieves (4 Å) in anhydrous MeOH (2.0 ml) and stirred at room temperature for 24 h. The reaction mixture was filtered, evaporated in vacuo and the residue separated with flash chromatography (CH₂Cl₂/MeOH—9:1) to leave compound 23 as a white solid (0.17 g, 49.5%). ¹H NMR (DMSO-_{d6}): δ 12.1 (br s, 1H), 7.81 (d, 1H), 7.29 (d, 1H), 7.14-7.07 (m, 2H), 4.51 (t, 2H), 4.10 (t, 2H), 4.01 (d, 2H), 3.06 (br d, 2H), 2.91-2.85 (m, 2H), 2.26-2.23 (p, 2H), 2.13-2.02 (m, 2H), 1.90-1.63 (m, 5H), 1.39-1.13 (m, 6H). MS (ES): 451.1 [M+H]⁺

### Compound 24

Following the procedure outlined for compound 23, N-(4-piperidylmethyl)-3,4-dihydro-2H-[1,3]oxazino[3,2-a]indole-10-carboxylate (0.20 g, 0.64 mmol) and 3-oxo-1-cyclopentane carboxylic acid (0.090 g, 0.69 mmol) was converted to compound 24 as a white solid (0.12 g, 44.1%). ¹H NMR (DMSO-_{d6}): δ 12.1 (br s, 1H), 7.81 (d, 1H), 7.29 (d, 1H), 7.15-7.06 (m, 2H), 4.49 (t, 2H), 4.11 (t, 2H), 4.01 (d, 2H), 2.96 (br d, 2H), 2.64-2.48 (m, 2H), 2.28-2.23 (p, 2H), 2.00-1.87 (m, 3H), 1.78-1.58 (m, 8H), 1.29-1.25 (m, 2H). MS (ES): 427.5 [M+H]⁺

### Compound 25

Following the procedure outlined for compound 23, N-(4-piperidylmethyl)-3,4-dihydro-2H-[1,3]oxazino[3,2-a]indole-10-carboxylate (0.20 g, 0.64 mmol) and 3-oxo-1-cyclohexane carboxylic acid (0.099 g, 0.69 mmol) was converted to compound 25 as a white solid (0.11 g, 39.4%). ¹H NMR (DMSO-_{d6}): δ 12.1 (br s, 1H), 7.83 (d, 1H), 7.28 (d, 1H), 7.16-7.06 (m, 2H), 4.50 (t, 2H), 4.11 (t, 2H), 4.02 (d, 2H), 2.89-2.83 (m, 2H), 2.61-2.12 (m, 5H),1.97-1.36 (m, 8H), 1.28-1.11 (m, 6H). MS (ES): 441.5 [M+H]⁺

### Compound 26

Following the procedure outlined for compound 1, N-(4-piperidylmethyl)-3,4-dihydro-2H-[1,3]oxazino[3,2-a]indole-10-carboxylate (0.31 g, 0.88 mmol) and methyl 2-[4-(bromomethyl)phenyl]propanoate (0.23 g, 0.88 mmol) was converted to compound 26 as a white solid (0.26 g, 61.2%). ¹H NMR (CDCl₃): δ 7.99 (d, 1H), 7.30-7.15 (m, 7H), 4.55 (t, 2H), 4.21 (d, 2H), 4.12 (t, 2H), 3.76-3.69 (q, 1H), 3.67 (s, 3H), 3.49 (br s, 2H), 2.92 (br d, 2H), 2.39-2.34 (p, 2H), 2.05-1.67 (m, 6H), 1.51 (d, 3H), 1.47-1.14 (m, 2H).

### Compound 27

Following the procedure outlined for compound 6, compound 26 (1.10 g, 2.24 mmol) was converted to the free acid 27 as a white solid (0.57, 53.5%). ¹H NMR (DMSO-_{d6}): δ 12.3.8 (br s, 1H), 7.79 (d, 1H), 7.55 (d, 2H), 7.33-7.14 (m, 3H), 7.13-7.08 (m, 2H), 4.50 (t, 2H), 4.20-4.03 (m, 6H), 3.76-3.70 (m 1H), 3.12-3.01 (m, 3H), 2.27-2.24 (m, 2H), 2.05-2.00 (m, 3H), 1.93-1.87 (m, 2H), 1.36 (d, 3H).

### Compound 28

Following the procedure outlined for compound 1, N-(4-piperidylmethyl)-3,4-dihydro-2H-[1,3]oxazino[3,2-a]indole-10-carboxylate (0.47 g, 1.5 mmol) and methyl 2-[4-(bromomethyl)phenyl]-2-methylpropanoate (0.40 g, 1.5 mmol) was converted to compound 28 as a white solid (0.34 g, 44.9%). ¹H NMR (CDCl₃): δ 7.97-7.92 (m, 1H), 7.25-7.10 (m, 7H), 4.51 (t, 2H), 4.17 (d, 2H), 4.08 (t, 2H), 3.63 (s, 3H), 3.47 (s, 2H), 2.92-2.86 (m, 2H), 2.37-2.29 (p, 2H), 2.02-1.92 (m, 2H), 1.91-1.66 (m, 3H). 1.55 (s, 6H), 1.43-1.38 (m, 2H).

### Compound 29

Following the procedure outlined for compound 6, compound 27 (0.34 g, 0.67 mmol) was converted to the free acid 28 as a white solid (0.15 g, 45.4%). ¹H NMR (DMSO-_{d6}): δ 12.10 (br s, 1H), 7.83-7.78 (m, 1H), 7.31-7.13 (m, 5H), 7.12-7.06 (m, 2H), 4.48 (t, 2H), 4.13-4.01 (m, 4H), 3.70 (br s, 2H), 2.84-2.80 (m, 2H), 2.26-2.21 (p, 2H), 1.96 (t, 2H), 1.91-1.72 (m, 3H), 1.44 (s, 6H), 1.32-1.22 (m, 2H).

### Compound 30

4-chloromethyl-5-methyl-1,3-dioxol-2-one (0.078 g, 0.53 mmol) was added to a mixture of free acid compound 24 (0.20 g, 0.40 mmol) and K₂CO₃ (0.16 g, 1.22 mmol) in DMA (1.0 ml). The mixture was stirred at room temperature for 12 hours and the mixture evaporated in vacuo. The residue was separated by flash chromatography (SiO₂, CH₂Cl₂:MeOH—9:1) to leave the prodrug 30 as a white solid (0.12 g, 50.0%). The corresponding hydrochloride salt was prepared.¹H NMR (DMSO-_{d6}): δ 10.30 (br s, 1H), 7.80 (d, 1H), 7.58-7.52 (m, 2H), 7.37-7.28 (m, 2H), 7.15-7.06 (m, 2H), 4.97 (s, 2H), 4.50 (t, 2H), 4.21 (d, 2H), 4.11 (t, 2H), 4.04 (d, 2H), 3.31 (br s, 2H), 2.94-2.91 (m, 2H), 2.26-2.22 (m, 2H), 2.11 (s, 3H), 1.93-1.89 (m, 3H), 1.63-1.55 (m, 2H), 1.51 (s, 6H).

### EXAMPLE 5

### In vitro Biological Testing of Hydrophilic 5-HT₄ Ligands in Binding Assays and Adenylyl Cyclase Assays

Materials and Methods

Establishment of HEK293 Cell Lines Stably Expressing Human 5-HT_{4(b)} Receptors

The development of HEK293 cell lines stably expressing human 5-HT_{4(b)} receptors was described and published previously (Bach et al. 2001). Briefly, HEK293 cells (ATCC) were grown in Dulbecco's modified Eagle's medium with 10% foetal calf serum and penicillin (100 U/ml) and streptomycin (100 μg/ml). Cells were transfected with plasmid DNA (pcDNA3.1(−) containing human 5-HT_{4(b)} receptor cDNA) using SuperFect Transfection Reagent (QIAGEN) according to the manufacturers protocol. Serial dilutions of transfected cells were plated in 96 well plates containing G418 (geneticin; Amersham) at 0.4 mg/ml, and isolated single colonies of cells transformed to the neomycin-resistant phenotype were expanded and tested for expression of serotonin receptors by measuring serotonin-stimulated adenylyl cyclase activity (Themmen et al. 1993). Transformed cells were always grown in the presence of G418 (0.4 mg/ml). For binding and adenylyl cyclase analysis, stable cell lines were grown and maintained in UltraCULTURE™ general purpose serum-free medium (BioWhittaker, Walkersville, Md., USA), supplemented with L-glutamine (2 mM), penicillin (100 U/ml) and streptomycin (100 μg/ml).

Membrane Preparation for Radioligand Binding and Adenylyl Cyclase Assay

Membranes were prepared from stably transfected HEK293 cells cultured on 150-mm cell culture dishes and grown to 80% confluence in serum-free medium (UltraCULTURE™, BioWhittaker) with penicillin (10 U/ml) and 2 mM L-Glutamine (BioWhittaker). Cells were washed twice with 10 ml ice-cold HBSS, scraped with a rubber policeman in 10 ml ice-cold HBSS and collected by centrifugation at 800 g for 5 min at 4° C. The cell pellet was resuspended in 1 ml/dish ice-cold STE buffer (27% (w/v) sucrose, 50 mM Tris-HCl, pH 7.5 at 20° C., 5 mM EDTA) and homogenized with an Ultra-Turrax (IKA) homogenizer, using five 10 s bursts with 30 s cooling in ice-water between bursts. To remove nuclei, the homogenate was centrifuged at 300 g for 5 min at 4° C. and the supernatant was further centrifuged at 17000 g for 20 min at 4° C. and the supernatant removed. The crude membrane pellet was resuspended with ten strokes of tight fitting pestle B in a Dounce glass-glass homogenizer in 1 ml/dish ice-cold TE (50 mM Tris-HCl, pH 7.5 at RT, 5 mM EDTA). This procedure was repeated twice and the resuspended membranes were finally aliquotted and flash frozen in liquid nitrogen and stored at −70° C. until use.

Radioligand Binding Assay

Binding assays were performed on membranes of HEK293 cells stably expressing the human 5-HT_{4(b)} receptor (refs.) in 96-well, round-bottom microtiter plates with total reaction volumes of 50-200 μl, containing the indicated concentration of [³H]GR113808 with or without competing unlabelled ligand in a binding buffer containing 50 mM Tris-HCl (pH 7.5 at RT), 1 mM EDTA, 5 mM EGTA, 2 mM MgCl₂, 1 mM ascorbate, 0.1% BSA and 100 μM GTP. The plates were incubated at 23 EC for 60 min and harvested onto UniFilter™-96 GF/C™ (Packard Instrument Co., Meriden, Conn., USA), presoaked in 0.3% polyethyleneimine (Sigma), using a Packard FilterMate Universal Harvester with 96-well format, and washed 4-6 times with approximately 0.25 ml/well of ice-cold buffer, containing 50 mM Tris-HCl (pH 7.0 at RT) and 2 mM MgCl₂. The filters were dried and counted at approximately 40% efficiency in a Top-Count liquid scintillation counter (Packard), using 20 μl per filter well of Micro-Scint liquid scintillation cocktail (Packard).

Adenylyl Cyclase Assay

Adenylyl cyclase activity was measured in membranes of HEK293 cells stably expressing the human 5-HT_{4(b)} receptor (refs.) by determining conversion of [α-³²P]ATP to [³²P]cAMP in membranes prepared in STE by homogenization of cells grown and washed as described above in a Dounce glass-glass homogenizer by 10 strokes with the tight-fitting pestle. Membranes were kept on ice prior to assay. Adenylyl cyclase activities were measured in 10-μl aliquots in a final volume of 50 μl in the presence of 0.1 mM [α-³²P]ATP (1-2×10⁶ cpm/assay), 4 mM MgCl₂, 20 μM GTP, 1 mM EDTA, 1 mM [³H]cAMP (ca. 10,000 cpm/assay), 1 μM 3-isobutyl-1-methyl xanthine (IBMX; Sigma), a nucleoside triphosphate regenerating system consisting of 20 mM creatine phosphate (Sigma), 0.2 mg/ml creatine phosphokinase (Sigma) and 40 U/ml myokinase (Sigma) and additives described in the text and figures. When forskolin (Calbiochem, La Jolla, Calif., USA) was used the concentration was 100 μM. Incubations were for 20 min at 32° C. Cyclic AMP formed was quantified by the double column chromatography system of Salomon et al. (1974) as modified by Bockaert et al. (1976).

Analysis of Binding and Adenylyl Cyclase Data

Binding and adenylyl cyclase data were analyzed by non-linear regression using Microsoft Excel with the Solver add-in, using the below equations.

Competitive binding assays—The data were fit to the equation

Y=a+(b−a)/(1+x/c) [1]

where a is non-specific binding, b is total binding in the absence of competitor, c is IC₅₀, and x is the concentration of competitor. Where relevant, relative binding data were obtained by recalculating the data using a=0 and b=100.

Activation of adenylyl cyclase—The data were fit to the equation

Y=a+(b−a)x/(c+x) [2]

where a is basal adenylyl cyclase activity, b is maximal adenylyl cyclase activity stimulated by the agonist, c is EC₅₀, and x is the concentration of agonist.

IC₅₀ values from competitive binding assays were converted to K_{b} values by the method of Cheng and Prusoff (1973).

Protein Measurements

The protein concentrations in the membrane preparations were measured with the Micro BCA Protein Assay Reagent Kit (Pierce, Rockford, Ill., USA) using bovine serum albumin (BSA) as standard.

Radiochemicals

[³H]GR113808 (84 Ci/mmol), [α-³²P]ATP (400 Ci/mmol) and [³H]cAMP (30-50 Ci/mmol) were from Amersham (Buckinghamshire, England).

Compounds

5-Hydroxytryptamine hydrochloride (5-HT, serotonin) was from Sigma (St. Louis, Mo., USA). GR113808 (1-methyl-1H-indole-3-carboxylic acid, [1-[2-[(methylsulfonyl)amino]ethyl]-4-piperidinyl]methyl ester) maleate was from Tocris (Avonmouth, UK). The other compounds tested were synthesized by Drug Discovery Laboratories AS (DDL) (Oslo, Norway).

Results of in vitro biological testing of 5-HT₄ ligands in adenylyl
cyclase and binding assays, organised by compound (Table 1)

| | Antagonist | | Agonist/ | Binding affinity | |
|---|---|---|---|---|---|
| | pK_{b} value | | Antagonist | (pK_{d} value) | |
| Compound | pK_{b} | N | properties | pK_{d} | n |
| GR113808 | 10.05 | 1 | Antagonist | 9.95-10.41 | 2 |
| SB207266 | 10.27-10.43- | 3 | Antagonist | 9.58-9.68-9.78- | 5 |
| (piboserod) | 9.57 | | | 10.45-10.39 | |
| 2 | 10.37-9.08 | 2 | Antagonist | 8.41-8.38-8.36 | 3 |
| 4 | 11.35 | 1 | Antagonist | ND | |
| 5 | ND | | | ND | |
| 6 | ND | | | ND | |
| 7 | 10.88-10.32 | 2 | Antagonist, | 11.60-12.69 | 2 |
| | | | Inverse | | |
| | | | agonist | | |
| 8 | 10.20-9.48 | 2 | Antagonist, | 10.44-10.11 | 2 |
| | | | Inverse | | |
| | | | agonist | | |
| 11 | | | ND | 6.96-7.26 | 3 |
| 12 | | | ND | 5.47 | 2 |
| 14 | | | ND | 6.59-6.81 | 3 |
| 17 | | | ND | 8.43-8.30 | 2 |
| 18 | | | ND | 6.76-6.90 | 2 |
| 20 | | | ND | 7.73-7.56 | 3 |
| 23 | 8.44 | 2 | Antagonist | 8.82 | 2 |
| 24 | 8.60 | 4 | Antagonist | 8.94 | 5 |
| 25 | 8.60 | 3 | Antagonist | 8.92 | 2 |
| 27 | 8.79 | 2 | Antagonist | 9.29 | 2 |
| 29 | 8.78 | 8 | Antagonist | 9.45 | 9 |

ND = not determined

### EXAMPLE 6

### Effects of Compounds 2, 7, 8, 24 and 27 on hERG (Huntigdon)

The purpose of this study was to assess the effects of the above compounds on hERG (human ether-a-go-go related gene) tail current by examining the acute effect of the test compounds on the hERG ion channel in an appropriate in vitro test system. The human embryonic kidney cell (HEK-293), which has been stably transfected with hERG ion channel cDNA, is a preparation that is considered suitable for this purpose.

Four concentrations of each test compound were tested in a screening assay to determine the liability to block hERG channels. Using the patch-clamp technique, peak hERG tail current amplitude was measured prior to and following exposure to the compounds at the following nominal concentrations:

Compound 2—10 nM, 1 μM, 10 μM, 30 μM and 50 μM;

Compound 7—100 nM, 1 μM and 10 μM;

Compound 8—100 nM, 1 μM, 10 μM and 100 μM;

Compound 24—100 nM, 1 μM, 10 μM and 100 μM; and

Compound 27—100 nM, 1 μM, 10 μM and 100 μM

for approximately 7 to 32 minutes (n=3 cells for each concentration). In addition, peak hERG tail current amplitude was measured in a separate group of 3 cells, prior to and following exposure to vehicle (0.1% DMSO) for time-matched vehicle data correction. Terfenadine (at the submaximally effective concentration of 50 nM, n=3), a known inhibitor of the I_{Kr} current, was used as a positive control compound.

The test compound data were corrected for the mean effect of vehicle and rundown and the concentration-response data were plotted and fitted with a sigmoidal function, from which the IC₅₀ values for each compound were calculated.

The IC₅₀ values for compounds 2, 7, 8, 24 and 27 were calculated to be 27.5 μM, 131 nM, 47.5 μM, 209 μM and 25 μM respectively.

1. A 5-hydroxytryptamine (5-HT) receptor modulating compound of formula I:
HT-L-A (I)
wherein:
HT is a 5-HT receptor modulating moiety containing a basic nitrogen atom;
A is an acid moiety; and
L is a linker moiety serving to maintain said basic nitrogen atom and said acid moiety at a separation of at least 0.4 nm,
or a prodrug form or salt thereof.

2. A compound as claimed in claim 1, wherein said acid moiety is a protic acidic moiety having a labile proton which, when in said acid moiety, is kept distanced from said basic nitrogen atom by said linker moiety by at least 0.6 nm.

3. A compound as claimed in claim 1, wherein the acid moiety A is selected from the group consisting of —C(O)—OR¹, —OP(O)OR²OR², —P(O)OR²OR², —SO₂OR², —SO₃H, —OSO₃H and —PO₃H; wherein R¹ and R² are independently selected from the group consisting of H, M (wherein M is a counter-ion), C₁₋₁₅-alkyl, C₃₋₈-cycloalkyl, aryl, and R^{1,2} wherein R^{1,2} is R′—O—C(O)R″, R′—O—C(O)—O—R″, R′—C(O)—O—R″, wherein R′ and R″ are independently selected from the group consisting of C₁₋₁₅-alkyl, C₃₋₈-cycloalkyl and aryl.

4. A compound as claimed in claim 1, wherein L comprises: an optionally substituted mono- or bi-cyclic aryl or heteroaryl group; a linear C₁₋₆-alkyl group being substituted independently at each carbon atom by at least one optionally substituted C₁₋₆-alkyl, C₂₋₆-alkenyl, C₂₋₆-alkynyl, C₃₋₁₀-cycloalkyl, aryl, heteroaryl, nitrile, hydroxy, amide, chloride or iodide group; an optionally substituted C₃₋₁₀-cycloalkyl or C₄₋₁₀-cycloalkenyl group; or an optionally substituted polycyclic alkyl or alkenyl group.

5. A compound as claimed in claim 1 wherein L is other than —CH₂-p-phenylene and —CO-p-phenylene.

6. A compound as claimed in claim 1, wherein L comprises a group of the formula —(CH₂)ₙ—Ar′—(CR^{a}R^{b})ₘ— in which n is 0 or 1; Ar′ is an optionally substituted aryl ring or heteroaromatic ring; R^{a} and R^{b} are each independently H or optionally substituted C₁₋₆-alkyl; and m is 0 or 1.

7. A compound as claimed in claim 1, wherein L is an optionally substituted, optionally bridged C₄-C₁₀-cycloalkyl group.

8. A compound of formula I as claimed in claim 1, wherein A is an oxyacid or a tetrazole group, or an acid or ester or salt thereof.

9. A compound as claimed in claim 1, herein HT is a group of formula II:
Ar—(C(O))ₙ-(E)ₘ-(G)ₚ-BN— (II)
wherein:
Ar is an optionally substituted aryl ring optionally fused with one or more rings selected from: non-aromatic, optionally substituted, carbocylic rings; non-aromatic heterocyclic rings; carbocyclic aromatic rings; and heteroaromatic rings;
n is 0 or 1;
m is 0 or 1;
E is 0 or NH;
p is 0 or 1;
G is a C₁₋₆-alkyl, C₃₋₇-cycloalkyl, C₁₋₆-alkyl-C₃₋₇-cycloalkyl or C₃₋₇-cycloalkyl-C₁₋₆-alkyl group; and
BN is a basic nitrogen moiety.

10. A compound as claimed in claim 1, wherein HT is a group of the formula III:
Ar—C(O)-E-G-BN— (III)
wherein:
Ar is a monocyclic or polycyclic aromatic or heteroaromatic;
E is selected from the group consisting of O and NH;
G is selected from the group consisting of C₁₋₆-alkyl, C₃₋₇-cycloalkyl, C₁₋₆-alkyl-C₃₋₇-cycloalkyl and C₃₋₇-cycloalkyl-C₁₋₆-alkyl; and
BN is a basic nitrogen moiety;
or wherein G-BN together form a C₃₋₇-heteroalkyl, or a C₁₋₆-alkyl-C₃₋₇-heteroalkyl group.

11. A compound as claimed in claim 1 wherein HT is a group having the formula IV: 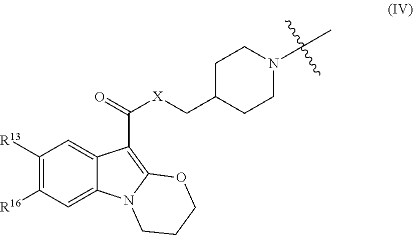
wherein:
R¹³ is selected from the group consisting of H, halogen, NH₂ and C₁₋₆-alkyl; and
R¹⁶ is selected from the group consisting of H, halogen, OH, O—C₁₋₆-alkyl and C₁₋₆-alkyl.

12. A compound as claimed in claim 1, wherein HT is a group of formula V: 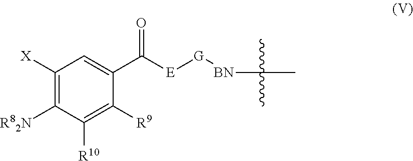
wherein:
E is selected from the group consisting of O and NH;
G is selected from the group consisting of C₁₋₆-alkyl, C₃₋₇-cycloalkyl, C₁₋₆-alkyl-C₃₋₇-cycloalkyl and C₃₋₇-cycloalkyl-C₁₋₆-alkyl;
BN is a basic nitrogen moiety;
or wherein G-BN together form a C₃₋₇-heteroalkyl, or a C₁₋₆-alkyl-C₃₋₇-heteroalkyl group;
X is a halogen;
R⁸ is independently selected from H and C₁₋₆-alkyl;
R⁹ and R¹⁰ are independently selected from the group consisting of H, O—C₁₋₆-alkyl, C₁₋₆-alkyl, a C₃₋₇-cycloalkyl, a heterocycloalkyl, a heteroaryl, or an aryl;
or wherein together R⁹ and R¹⁰ form a C₃₋₇-cycloalkyl, a heterocycloalkyl, a heteroaryl, or an aryl;
or wherein NR⁸₂ and R¹⁰ together form a heterocycloalkyl group.

13. A compound of formula I as claimed in claim 1, wherein L-A is a group of formula VII 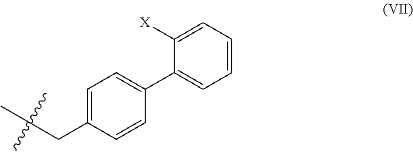
wherein:
X is —C(O)OH, optionally substituted —C(O)O—C₁₋₆-alkyl or an optionally substituted 5-tetrazolyl group,
or a prodrug form or salt thereof.

14. A compound of formula Ib:
HT-L_{b}-A_{b} (Ib)
wherein:
HT is a group having 5-HT receptor modulating activity, wherein HT is a 5-HT receptor modulating moiety containing a basic nitrogen atom;
A_{b} is a group having renin-angiotensin system modulating activity; and
L_{b} is absent or is a linker which enables the pharmacophores of HT and A_{b} to function,
or a prodrug and/or salt thereof.

15. The compound of claim 14, wherein A_{b} denotes a group of formula VII: 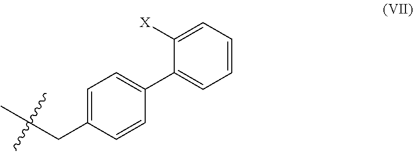
wherein:
X is —C(O)OH, optionally substituted —C(O)O—C₁₋₆-alkyl or an optionally substituted 5-tetrazolyl group.

16. A pharmaceutical composition comprising a 5-HT receptor modulating compound as claimed in claim 1, or a physiologically tolerable prodrug form or salt thereof, together with at least one pharmaceutical carrier or excipient.

17. (canceled)

18. (canceled)

19. (canceled)

20. A method of treatment of a disease of the cardiovascular system, the gastrointestinal system or the urinary system comprising administering an effective amount of a 5-HT receptor modulating compound as claimed in claim 1, or a physiologically tolerable prodrug form or salt thereof, to a patient in need thereof.

21. The compound of claim 1, wherein L is a linker moiety serving to maintain said base nitrogen atom and said acid moiety at a separation of at least 0.5 nm.

22. The compound of claim 1, wherein L is a linker moiety serving to maintain said base nitrogen atom and said acid moiety at a separation of at least 0.6 nm.

23. The compound of claim 1, wherein L is a linker moiety serving to maintain said base nitrogen atom and said acid moiety at a separation of at least 0.65 nm.

24. The compound of claim 1, wherein L is a linker moiety serving to maintain said base nitrogen atom and said acid moiety at a separation of at least 2 nm.

25. A compound as claimed in claim 4, wherein said polycyclic alkyl or alkenyl group has a steroid backbone.

26. A compound as claimed in claim 6, wherein n is 1.

27. A compound as claimed in claim 6, wherein R^{a} and R^{b} are each independently optionally substituted C₁₋₆-alkyl.

28. A compound as claimed in claim 6, wherein R^{a} and R^{b} are each independently optionally substituted C₁₋₄-alkyl.

29. A compound as claimed in claim 6, wherein R^{a} and R^{b} are each independently optionally substituted methyl.

30. A compound as claimed in claim 6, wherein m is 1.

31. A compound as claimed in claim 1, wherein L is a C₅-C₈-cycloalkyl group.

32. A compound as claimed in claim 1, wherein L is a C₅-C₇-cycloalkyl group.

33. The compound of claim 9, wherein BN is a basic nitrogen atom-containing moiety selected from an amine group, an amide group, a carbamate or a carbamate derivative, urea or a urea derivative, a carbazimidamide, a nitrogen-containing heterocyclic ring, a nitrogen-containing heteroarylic ring, and an azabicyclic ring.
